(19) United States
(12) Patent Application Publication
(10) Pub. No.: US 2008/0269432 A1
(43) Pub. Date: Oct. 30, 2008

(54) Device For Injecting Successive Layers Of Fluid In A Circulating Fluidised Bed And Methods Using Same

(75) Inventor: Alex de Broqueville, Grez-Doiceau (BE)

Correspondence Address: FINA TECHNOLOGY INC, PO BOX 674412, HOUSTON, TX, 77267-4412, US

(73) Assignee: TOTAL PETROCHEMICALS RESEARCH FELUY, Seneffe (Feluy) (BE)

(21) Appl. No.: 12/066,609

(22) PCT Filed: Sep. 15, 2006
(86) PCT No.: PCT/EP2006/066404

§ 371 Date: Jul. 14, 2008

(30) Foreign Application Priority Data
Sep. 15, 2005 (BE) 2005/0443

(51) Int. Cl. C08F 2/01; B01J 8/20; C07C 5/327; B05D 7/00

(52) U.S. Cl. 526/64; 422/140; 427/213; 585/440

(57) ABSTRACT

Device for injecting fluid in successive layers into a rotating fluidized bed and methods for catalytic polymerization, drying or other treatments of solid particles or for catalytic conversion of fluids, in which a succession of injectors (12), distributed around the fixed circular wall (2) of a circular reaction chamber, inject along this wall, in successive layers, one or more fluids (13), which entrain the solid particles (17), passing through this chamber, in a movement of rapid rotation whereof the centrifugal force concentrates these particles along this wall, thereby forming a fluidized bed rotating around a central duct (3), through which the fluids are removed.

The present invention relates to a device for injecting fluid, in successive layers, into a rotating fluidized bed, inside a fixed circular reaction chamber, and to methods for catalytic polymerization, drying, impregnation, coating or other treatments of solid particles in suspension in the rotating fluidized bed, or for cracking, dehydrogenation, or other catalytic conversions of fluids using this device.

To obtain a high concentration of solid particles in a conventional fluidized bed, subject to the force of gravity alone, the fluid passing through the fluidized bed must exert on the solid particles an upward pressure lower than the downward pressure of the solid particles due to the force of gravity, and its upward velocity must therefore be low, thereby limiting the fluid flow rate which can pass through the fluidized bed and the difference in velocity of the fluid to that of the solid particles in suspension in this fluid.

In a rotating fluidized bed, in which the centrifugal force may be substantially higher than the force of gravity, the centripetal pressure exerted by the fluid passing radially through the fluidized bed may be substantially higher and therefore its flow rate and its difference in velocity to that of the solid particles may both be substantially higher, thereby improving the contact between the fluid and the solid particles and substantially increasing the volume of fluid that can pass through the fluidized bed and therefore also its capacity to cool, heat and/or dry the solid particles.

If the rotating fluidized bed is supported by a fixed cylindrical wall along which it must slide, the pressure exerted by the solid particles against this fixed cylindrical wall slows these solid particles down to an extent commensurate with the thickness, density and rotational velocity of the fluidized bed. The rotational velocity will decrease rapidly if the angular momentum of rotation is not maintained using rotating mechanical means, with the problems associated with the presence of moving equipment inside the reactor, and/or by the injection of fluid at high velocity, in the direction of rotation of the fluidized bed. However, if the density of the fluid is much lower than that of the solid particles the quantity of fluid that must be injected to transfer the necessary angular momentum to the solid particles is very high and it may prevent the formation of a thick and dense fluidized bed and the proper separation of the fluid and the solid particles.

In fact, when a fluid is injected at high velocity, tangentially to the cylindrical wall and perpendicularly to the axis of symmetry of a cylindrical chamber traversed by a central duct comprising discharge openings for removing this fluid, the fluid can make several turns around this central duct before penetrating thereinto if the discharge openings are narrow. However, as soon as solid particles are introduced into this cylindrical chamber, they slow down the fluid to the extent commensurate with the ratio of the specific gravity of the solid particles to that of the fluid. Accordingly, the removal of the fluid becomes more direct, and this may even cause a reversal of the fluid flow along the central duct, downstream of the discharge openings, and generate turbulence which entrains the solid particles toward the outlet, thereby limiting the possibility of forming a thick and dense fluidized bed inside the cylindrical chamber.

The present invention relates to a rotating fluidized bed device comprising a circular reaction chamber, a device for feeding one or more fluids, placed around the circular wall of said cylindrical reaction chamber, a device for removing said fluid or fluids, a device for feeding solid particles on one side of said circular reaction chamber and a device for removing said solid particles on the opposite side of said circular reaction chamber,

characterized in that:
said device for removing said fluid or fluids comprises a central duct passing longitudinally through or penetrating into said reaction chamber, the wall of said central duct comprising at least one discharge opening for centrally removing, via said central duct, said fluid or fluids from said circular reaction chamber;
said device for feeding said fluid or fluids comprises fluid injectors distributed around said circular wall for injecting said fluid or fluids in a succession of layers along said circular wall and rotating around said central duct while entraining said solid particles in a rotational movement whereof the centrifugal force thrusts them toward said circular wall through said succession of layers;
said centrifugal force is, on average, at least equal to three times the force of gravity, said solid particles thereby forming a rotating fluidized bed which rotates around and at a certain distance from said central duct while sliding along said circular wall and while being supported by said layers of said fluid(s) which pass through said fluidized bed before being removed centrally via said discharge opening of said central duct and whereof the centripetal force is offset by said centrifugal force exerted on said solid particles.

In the present invention, injectors, distributed around the circular wall of a circular reaction chamber, inject one or more fluids, along the circular wall, in successive layers, to form a succession of fluid layers which are superimposed and rotate rapidly inside the reaction chamber, around a central duct which penetrates thereinto or passes through it along its central axis, and which is provided with one or more discharge openings through which the fluid can be removed centrally. The circular reaction chamber is traversed by a stream of solid particles which are fed at one of its sides and removed at the opposite side and which are entrained by the fluid in a rapid rotational movement whereof the centrifugal force is sufficient to concentrate them, before they leave the circular reactor chamber, in a dense rotating fluidized bed, which is at least partially supported by the centripetal pressure of these successive fluid layers moving along the circular wall and which act as fluid pads, reducing the friction of the solid particles against this wall. The fluid is fed by a feed device which may comprise a fluid feed chamber surrounding the circular reaction chamber, the pressure difference, preferably higher than the average pressure due to the centrifugal force of the rotating fluidized bed against the circular wall, between the feed device and the central duct, and the flow rate of the fluid(s) suitable for supporting and rotating the fluidized bed at a velocity generating a substantial average centrifugal force, preferably greater than three times the force of gravity.

To avoid the entrainment of the solid particles in the central duct, the velocity and/or difference between the fluid injection and removal must be greater, and the losses of angular momentum of rotation of the solid particles must be lower, as the radius of the reaction chamber and the ratio of the densities of the solid particles and the fluid increase.

For this purpose, to limit the pressure and concentration of solid particles against the circular wall of the reaction chamber and hence their slowdown, it is advisable that in each annular section of the reaction chamber, there should be at least one fluid injector at 90° intervals, that is 4, and preferably at least seven, the most preferable being at least 11, and hence that the number of successive fluid layers should be high, or that the distance between these injectors should be short, preferably shorter than the average radius of the circular chamber, to limit the quantity and concentration of the solid particles coming into contact with this circular wall after having passed through the layer of fluid which has been injected by the injector located upstream, before reaching the fluid layer injected by the injector located downstream.

It is also advisable for the profile of the injectors to be designed in order to inject the fluid at a sufficient velocity, preferably at least twice the desired rotational velocity of the solid particles in the fluidized bed, and in thin layers, with a thickness at the time of their injection that is preferably less than one-twentieth of the average radius of the reaction chamber, in a direction making an acute angle, preferably of less than 30°, with the circular wall, and for the planes of the outlet openings of the fluid injectors to form, with the circular wall side located downstream, angles preferably of between 60° and 120°, so that the thrust of the fluid(s) at the time that they exit the injectors is more tangential than radial or centripetal. The circular wall may be cylindrical, but it may also have various radii of curvature or be plane between the fluid injectors. In the latter case, the circular wall is polygonal and its sides located on each side of the injectors make an angle that approaches 180° as the number of injectors increases.

It is also preferable, to facilitate the rotation of the fluid around the central duct and to reduce the possibility of a reversal of the fluid flow which may rise along the wall of the central duct downstream of the discharge openings, that no cross section of the central duct should comprise more than a single fluid discharge opening, and that these openings should be narrow, arranged longitudinally, preferably having an average width lower than half of the average distance between the central duct and the circular wall, and that the sum of the cross sections of the discharge openings should preferably be lower than twice the sum of the cross sections of the outlet openings of the fluid injectors, which is itself preferably lower than half of the average longitudinal cross section of the circular reaction chamber, and that the planes of these discharge openings should make, with the wall of the central duct, an angle preferably of between 60° and 120°, this wall progressively deviating from the circular wall of the reaction chamber, from its side located downstream of the discharge openings to the opposite side, thereby having the appearance of a spiral.

The present invention may comprise at least one deflector, wing-shaped, passing longitudinally through the reaction chamber, close to the wall of the central duct, having its leading edge upstream of the fluid removal opening(s) and its trailing edge downstream of these fluid removal openings, in order to reintroduce, into the reaction chamber, the solid particles, generally the finest, which have entered the space located between the deflector and the wall of the central duct. The inlet cross section of this space is preferably larger than the sum of the cross sections of the discharge openings, and the distance between the trailing edge and the wall of the central duct is preferably less than half of the distance between this edge and the circular wall. This deflector may be hollow and provided with fluid injectors arranged along its trailing edge, in order to inject a thin layer of fluid at high velocity, approximately parallel, and preferably at approximately 30° to the wall of the central duct, downstream of the discharge openings, in order to prevent these solid particles from rising along the wall of the central duct downstream of the discharge opening.

The present invention may comprise at least one transverse control ring, which is placed close to the outlet of the solid particles, whereof the outer edge extends along and is fixed to the circular wall and whereof the inner edge surrounds and is at an average distance from the central duct, preferably greater than one-quarter of the average distance between the central duct and the circular wall, to enable the solid particles to pass from one side of the fluidized bed to the other without coming too close to the discharge openings of the central duct. This control ring serves to prevent or to slow down the transfer of the solid particles from upstream of this ring to downstream thereof, as long as the fluidized bed has not reached the desired thickness upstream. This ring may comprise a passage along the circular wall, to permit a sufficient minimum passage to progressively drain the circular reaction chamber when the solid particle feed is stopped.

The present invention may comprise a set of helical turns, whereof the outer edges extend along and are fixed to the circular wall and whereof the inner edges surround and are at an average distance from the central duct, preferably greater than one-quarter of the average distance between the central duct and the circular wall, to enable the solid particles traveling longitudinally in one direction, when they extend along these helical turns, to travel in the other direction in the space between these helical turns and the central duct, without too closely approaching the discharge openings of the central duct. These helical turns, which may form a continuous or discontinuous helical spiral or may be fragmented into a set of fins, are suitable for making the solid particles pass from one side to the other of the circular reaction chamber numerous times and/or for making them rise longitudinally, if the axis of rotation of the fluidized bed is inclined or vertical. Similar devices are described in Belgian patent applications Nos. 2004/0186 and 2004/0612, filed 14 Apr. and 12 Dec. 2004 in the name of the same inventor.

In the present invention, the axis of rotation of the fluidized bed may be horizontal, inclined or vertical.

If it is horizontal or inclined by less than 45°, preferably less than 30°, the average velocity of the solid particles, their concentration and the pressure they exert on the thin fluid layers are higher in the bottom of the reaction chamber. It is therefore preferable to divide the outer distribution chamber into several longitudinal segments by longitudinal separating walls in order to differentiate between the fluid injection pressure in the various fluid injectors according to their position in the reaction chamber.

If the axis of rotation of the fluidized bed is approximately vertical or inclined by more than 45°, preferably at least 60°, separating rings, surrounding the central duct at a certain distance therefrom, preferably less than one-third of the average distance between the circular wall and the central duct to enable the solid particles to pass into this space without too closely approaching the discharge opening of the central duct, may be fixed against the circular wall to prevent the excessively rapid drop of the solid particles. The pressure exerted by these solid particles against the upper surface of these separating rings slows down not only their fall, but also their rotational movement. This may be compensated for, if necessary, if these rings are hollow and provided with fluid injectors for injecting a fluid in thin layers along their upper surface in the direction of rotation of the solid particles.

In the present invention, these separating rings can be replaced by helical turns, which may also be hollow and which can form a continuous or discontinuous helical spiral or may be fragmented into fins, fixed against the circular wall, the orientation of the slope of the turns or the fins entraining the solid particles upward, said solid particles rapidly rotating along the circular wall, and the average distance between the inner edge of the turns and the central duct, preferably greater than one-quarter of the average distance between the circular wall and the central duct, enabling the solid particles, which have risen along the upper surface of these turns, to fall back into this space without too closely approaching the discharge opening in the central duct. This serves to feed the solid particles into the bottom of the circular reaction chamber and to remove them at the top. Similar devices are described in Belgian patent applications Nos. 2004/0186 and 2004/0612, filed 14 Apr. and 12 Dec. 2004 in the name of the same inventor.

In the present invention, the central duct may be traversed only on one side of the circular reaction chamber, preferably the upper side if the axis of rotation of the fluidized bed is vertical or inclined, and may terminate before reaching the opposite side.

Its cross section may decrease progressively and its end located in the circular reaction chamber may be open or closed.

In the present invention, the distribution chamber may be divided into successive annular sections by transverse annular separating walls in order to differentiate between the quality and quantity of the fluids which are fed to the different sections and which pass through the corresponding section of the rotating fluidized bed, and these fluids may be recycled to the same sections or to other sections, if the central duct is also divided into successive sections, connected to tubes passing inside the central duct and suitable for removing these fluids separately.

In the present invention, several circular reaction chambers may be installed in series, connecting the outlet of the solid particles from one chamber to the inlet of the solid particles of the next chamber, and the solid particles may be recycled, after having been regenerated, if they are catalytic, by an appropriate device after having spent a more or less long period of time, as required, in the circular reaction chamber(s). In another embodiment, the device according to the present invention is characterized in that said circular reaction chamber is connected to another similar chamber, by a transfer line for transferring said solid particles from said circular reaction chamber to said similar chamber and whereof the inlet is located close to said circular wall of said circular reaction chamber, on the side opposite said device for feeding said solid particles, and whereof the outlet is located close to said central duct of said similar chamber on the side opposite said device for removing said solid particles from said similar chamber. A similar device is described in Belgian patent application No. 2004/0612, filed 12 Dec. 2004 in the name of the same inventor.

The present invention is suitable for making a very large quantity of fluid pass through a dense rotating fluidized bed, with good separation between the solid particles and the fluid, and for making it rotate rapidly in order to obtain a high centrifugal force, without the use of rotating mechanical means inside the reactor, even if the fluid density is low. It permits easy recycling, after appropriate treatment, of the fluid and/or the solid particles, whereof the residence time can be adjusted as required. It is particularly advantageous for methods requiring very good contact between the fluid and the solid particles, like the rapid drying of solid particles in a compact reactor, and/or a high heat transfer capacity for controlling the temperature of highly exothermic catalytic reactions, like the catalytic polymerization of ethylene, or highly endothermic reactions like the catalytic dehydrogenation of ethylbenzene or the catalytic cracking of light gasolines. It is also suitable for the regeneration of catalyst particles at the desired rate, and the high rotational velocity of these solid particles reduces the probability of them forming aggregates or adhering to the reactor surface. The presence of fluid pads between the solid particles and the reactor surface also reduces the attrition of the solid particles and of the reactor walls.

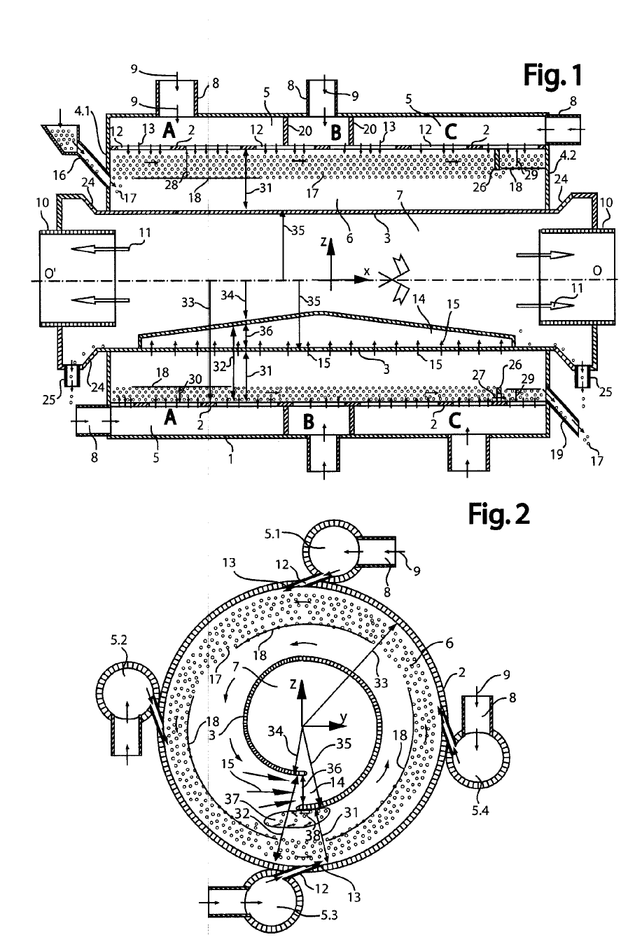

FIG. 1 shows the schematic longitudinal section, in the plane of the (x) and (z) axes, the (x) axis coinciding with the axis of rotation of the fluidized bed (00′) and the (z) axis, directed upward, coinciding with the vertical, of a cylindrical reactor comprising three concentric walls, the outer wall (1), the median wall, called the circular wall (2) and the central wall (3), called the wall of the central duct, the space comprised between the outer wall and the central wall being closed by two annular side walls (4.1) and (4.2).

The space (5) between the outer wall and the circular wall is the chamber for feeding fluid(s), the space (6) between the circular wall and the central wall is the circular reaction chamber, and the space inside the central wall is the central duct (7).

Tubes (8) are used for introducing the fluid(s), symbolized by the arrows (9) through the outer wall (1) or the annular side walls (4.1) and (4.2), into the feed chamber (5) and tubes (10) for removing the fluid(s), symbolized by the arrows (11), from the central duct (7). Longitudinal slits (12), which may extend continuously from one end of the circular reaction chamber to the other or, as is the case in this figure, may extend along more or less long lengths and be separated from one another by more or less long distances, passing through the circular wall (2), illustrate the fluid injectors for injecting, into the circular reaction chamber (6), the fluid(s), symbolized by the arrows (13), in thin layers, at high velocity, along the circular wall (2), and a discharge opening (14) in the wall of the central duct (3) serves to remove this fluid, symbolized by the arrows (15), from the circular reaction chamber (6) in the central duct (7). Since the fluid(s) rotate rapidly in the circular reaction chamber, the tangential component of their velocity is substantially higher than the radial component, but it is not visible because it is perpendicular to the plane of the figure.

A line (16) is available for introducing solid particles, symbolized by small circles (17), through the side wall (4.1). The solid particles are entrained by the fluid in a rotational movement and the centrifugal force maintains them along the circular wall (2) where they form a fluidized bed with an approximately cylindrical surface (18). A line (19) is use to remove the solid particles (17) through the opposite annular side wall (4.2).

Annular walls (20) can divide the distribution chamber (5) into annular sections (A), (B) and (C) in order to feed the fluid(s) having different properties and/or different pressures.

The tubes (10) for removing the fluid(s) may penetrate into the central duct (3) which is wider at its two ends, thereby forming kinds of cyclones. The solid particles, which have penetrated into the central duct and which rotate rapidly, concentrate along the conical walls (24), and are removed by the tubes (25) and optionally recycled.

The fluidized bed can be divided by a control ring (26) optionally provided with one or more passages (27) against the circular wall to enable the solid particles to pass from one side to the other. If the feed rate of the solid particles (17) via the line (16) is higher than the transfer rate of the solid particles through the passages (27), the thickness (28) of said fluidized bed upstream of the control ring (26) increases until it is sufficient for particles to overflow via the center of this ring to pass to the other side. And if the outlet flow rate of the solid particles via the line (19) is higher than the feed rate, the thickness (29) of the fluidized bed downstream of the control ring (26) decreases until the rarefaction of the solid particles automatically adjusts the outlet flow rate with the inlet flow rate of these particles. This device serves to maintain the volume of the fluidized bed approximately constant upstream of the control ring (26), preferably located close to the outlet (19), if the solid particle feed rate is sufficiently high. The passages (27) also serve to remove all the solid particles from the circular reaction chamber when the solid particle feed is stopped.

Since the reactor is horizontal, the effect of the force of gravity creates a difference in thickness of the fluidized bed and/or the solid particle concentration between the top (28) and the bottom (30) of the circular reaction chamber. The outlet (14) is preferably in the bottom of the reactor because the velocity and concentration of the particles is a maximum there, and hence the thickness of the fluidized bed is a minimum there, thereby reducing their probability of being entrained in the central duct (7).

Since the plane of the discharge opening (14) is perpendicular to the wall of the central duct, the thickness or width (31) of the reaction chamber is a minimum downstream of the discharge opening (14) and it is a maximum (32) upstream. The circular wall (2) is cylindrical in this illustration, and hence its radius (33) is constant, while the radius of curvature of the wall of the central duct (3) is variable. It is a minimum (34) upstream of the outlet (14) and a maximum (35) downstream.

The width (36) of the discharge opening (14) may be a maximum at the middle of the reaction chamber and a minimum close to the annular side walls (4.1) and (4.2) so that the cross section of the central duct is greater at its ends, in order to facilitate the removal of the fluid (11). It should be observed that this width (36) is preferably nil against these walls, to prevent the solid particles slowed down by these walls from being entrained into the central duct.

The reactor may be slightly inclined to increase the particle flow toward their outlet and hence to decrease their residence time in the reaction chamber. In this case, the surface of the fluidized bed is slightly conical, according to the extent of the inclination and the ratio of the force of gravity to the centrifugal force.

FIG. 2 shows the schematic cross section, along the plane of the (y) and (z) axes, of the reactor in FIG. 1, where the annular distribution chamber (5) is replaced by four tubular distribution chambers, from (5.1) to (5.4), each connected to a fluid injector or set of fluid injectors (12). This arrangement may be preferred when the number of injectors is small.

It should be observed that the radius of curvature (35) of the wall (3) of the central duct is smaller (34) at its part upstream of the discharge opening (14), giving the appearance of a spiral, and that the width (31) of the circular chamber is preferably smaller downstream than upstream (32), because the fluid flow rate rotating around the duct increases as it approaches the discharge opening (14).

The surface (37) illustrates the cross section of a turbulence zone generated by the optional reversal of the fluid flow, illustrated by the arrows (38), downstream of the outlet (14) of the central duct. This turbulence may cause the removal of solid particles, generally the finest, via the discharge opening (14).

It is useful to observe that the force of gravity, which adds to the centrifugal force in the bottom of the reactor and which increases the velocity of the solid particles therein and hence the centrifugal force, generates a higher pressure there against the circular wall, which can justify a higher injection pressure in the tubular distribution chamber (5.3). Moreover, it may be advisable to reduce the injection pressure of the tubular chamber (5.2), upstream of the discharge outlet (14), to decrease therein the centripetal pressure of the fluid on the solid particles and hence the risk of entraining them into the central duct.

The numerical simulation shows that it is possible, in a cylindrical chamber 40 cm in diameter with 4 fluid injectors, injecting air at atmospheric pressure in a direction making an angle of 30° with the cylindrical wall, distributed at 90° intervals around each annular section of the cylindrical chamber, to form a dense rotating fluidized bed. However, it has been found that a large quantity of solid particles passes through the thin fluid layers and is slowed down along the circular surface upstream of the injection slits, where their concentration approaches the theoretical maximum, thereby increasing the resistance to the rotation of the fluidized bed. It has also been found that the interaction between the solid particles, whereof the slowdown generates a high pressure upstream of the injectors and the fluid whereof the injection pressure must be high to offset this high pressure of the solid particles of the opening of the injector outlets, may locally generate a strong centripetal thrust, which may project the solid particles toward the discharge opening if this strong thrust is upstream of the discharge opening, and thereby cause losses of solid particles.

To reduce this braking effect and avoid resonance events which can cause losses of solid particles, it is advisable to increase the number of injectors, preferably a primary number, and/or for the distance between the injectors not to be identical throughout. It is also preferable to give the injectors and the circular wall a shape suitable for minimizing the centripetal thrust of the fluid and favoring its tangential thrust.

Thus in FIG. 2, the planes of the outlet openings of the injectors are virtually merged with the planes parallel to the circular surface which is cylindrical, thereby favoring the centripetal thrust due to the fluid pressure on the solid particles even if the fluid injection angle is small.

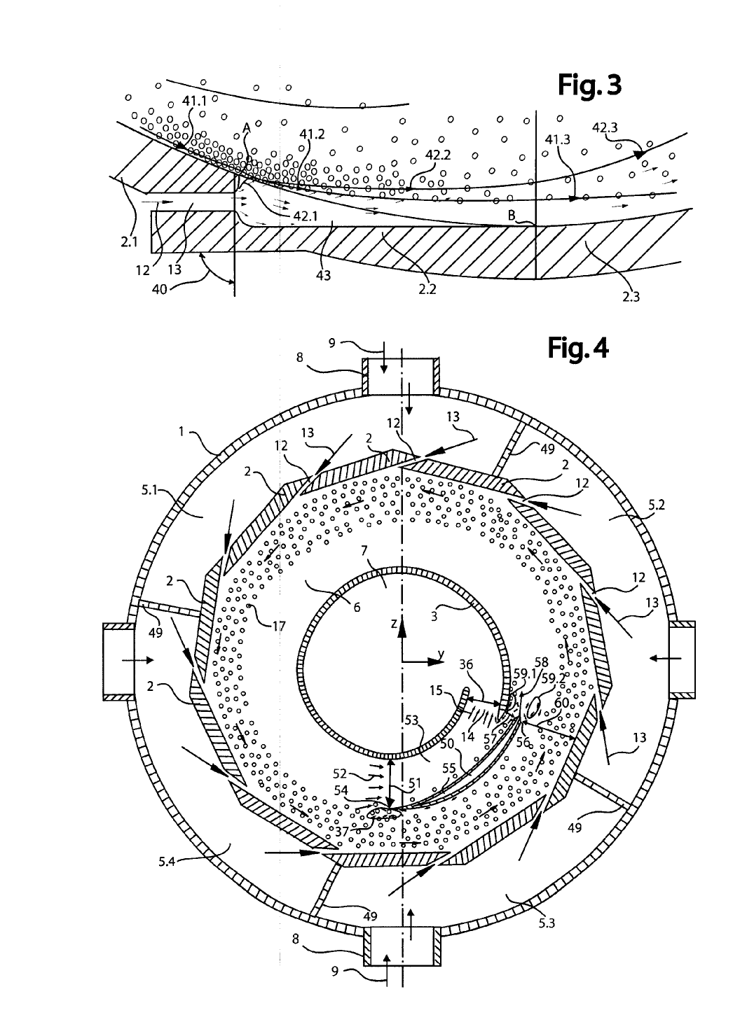

FIG. 3 shows the schematic cross section of the zone around a fluid injector, showing how a small modification of the circular wall (2.2) downstream of a fluid injector (12), the latter becoming plane and tangential, at (B), at the prolongation of the circular wall (2.3), changes the orientation of its outlet planer which accordingly makes an angle (40) of about 90° with the plane wall (2.2). The thrust generated by the high pressure of the fluid (13.1) on the upstream side of its outlet, at (A), is thereby directed more tangentially to the circular wall.

The solid particles, highly concentrated, symbolized by small circles (17), form a compact set which slides along the circular wall (2.1) in the direction (41.1) upstream of the injector (12.1). Their meeting with the flow line (42.1) of the fluid (13), at the injector outlet, deviates them progressively and accelerates them along the flow line (41.2) and hence their concentration decreases progressively, enabling an increasingly large fraction of the fluid to penetrate this less and less compact set of solid particles by following the fluid flow line (42.2) which penetrates increasingly (42.3) into the fluidized bed while deviating from the wall (2.3).

The fluid pressure in the space (43), between the wall (2.2) and the flow line (41.2) of the solid particles must be sufficient to prevent the solid particles from clogging the fluid outlet and therefore to deviate them along this flow line (41.2). As the fluid accelerates the solid particles, its energy and hence its pressure decreases, enabling the solid particles which follow the flow line (41.3) to approach the circular wall (2.3) which slows them down and hence increases their concentration until they pass in front of the next injector. And so on and so forth.

If the angle (40) between the plane of the injector outlet (12) and the circular wall was closer to 0° as in FIG. 2, the change in direction (41.2) of the solid particles would be more sudden, generating a higher pressure and hence a higher fluid thrust on the solid particles located against the part upstream of the injector, in a direction perpendicular to this plane and hence centripetal, and the flow line (41.2) would deviate more from the wall (2.2), thereby increasing the slowdown of the solid particles upstream and bringing them closer to the central duct.

This illustration shows how the solid particles slowed down by the curved wall of the reaction chamber and, striking the obstacle presented by the injection of a fluid jet, can form a compact set which substantially slows down the normal sliding of these solid particles, and how the arrangement and orientation of the outlet opening of the injectors and of the fluid injection direction can minimize this braking effect and the centripetal pressure exerted by the fluid on the solid particles upstream of its outlet.

FIG. 4 shows the schematic cross section, along the plane of the (y) and (z) axes of a reactor whereof the devices for feeding and removing the fluid(s) from the reaction chamber have been modified to improve the proportion between the transfer of the tangential and centripetal angular momentum of the fluid to the solid particles and to reduce the quantity of solid particles escaping via the discharge opening (14) of the central duct. The number of fluid injectors having been increased, 11 in this example, the feed chamber is preferably bounded by a cylindrical wall (1) surrounding the circular wall (2) and it is divided into longitudinal segments, from (5.1) to (5.4), by longitudinal walls (49), to feed the various fluid injectors (12) at different pressures.

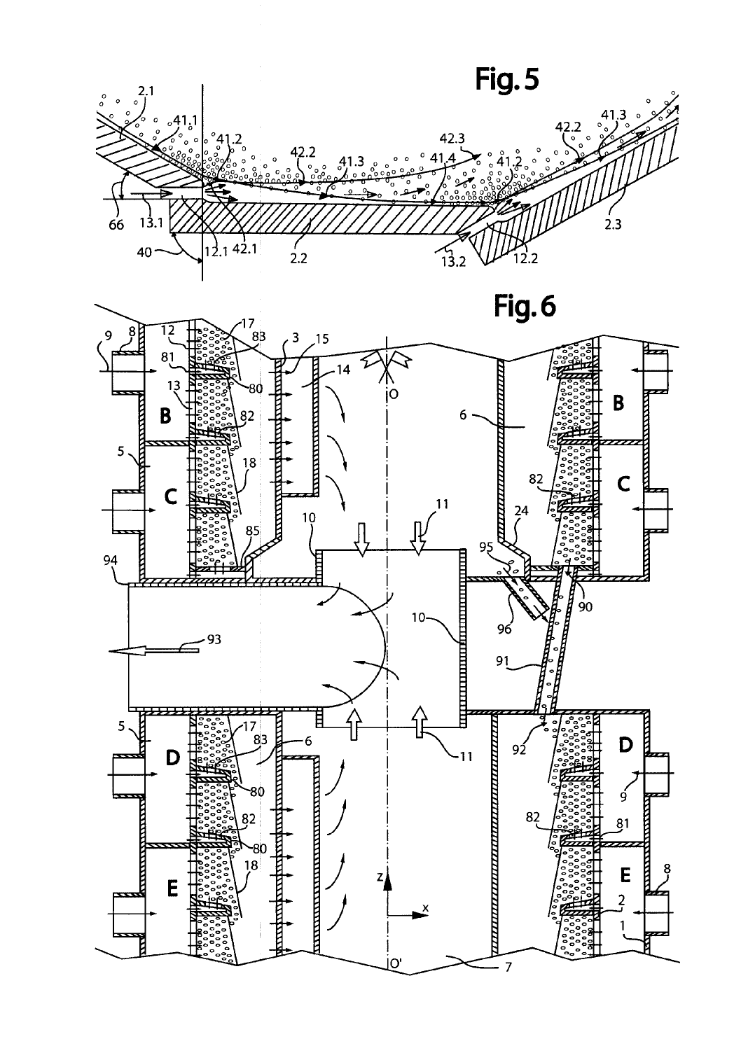

The circular wall is plane between two injectors (12). It is therefore polygonal. The fluid is injected parallel to this surface, according to the arrangement shown in FIG. 5, to facilitate the sliding of the solid particles along it and to reduce their concentration upstream of the injection slits and hence to decrease their resistance to travel.

A hollow wing-shaped deflector with a cross section (50), passing through longitudinally, that is perpendicular to the plane of the figure, the circular reaction chamber (6) and fixed to the two annular side walls (4.1) and (4.2), not shown in this figure, through which a pressurized fluid can be introduced therein, is placed at a distance (51) from the wall of the central duct (3), upstream of the discharge opening (14). It channels the fluid stream (52) into the space (53) between it and the wall of the central duct.

The turbulence zone (37) which may develop along the leading edge (54) of the deflector (50) can entrain solid particles into this space (53). The distance (51) being preferably greater than the thickness (36) of the discharge opening (14), the velocity of the fluid (52) which accelerates these solid particles, progressively increases and the centrifugal force thrusts them along the curved inner wall (55) of the hollow deflector (50).

The trailing edge (56) of the deflector, located at the distance (57) from the wall of the central duct (3), is equipped with one or more fluid injectors for injecting a thin layer of fluid (58) at high velocity more or less parallel, preferably at less than about 30°, to the wall of the central duct (3) producing a suction effect which returns to the reaction chamber (6) beyond the discharge opening (14), the solid particles flowing along the inner wall (55) of the deflector. However, a turbulence zone (59.1) may be developed between the thin fluid layer (58) and the wall of the central duct (3) and generate a flow reversal which returns part of these particles to the outlet (14). To minimize this influence, it is preferable for the pressure drop in the space (53) to be low and hence for the quantity of solid particles that the fluid stream (52) must accelerate to be low and for the distance (57) to be short, preferably shorter than half of the distance (60) between the leading edge and the circular wall.

Another turbulence zone (59.2) may be developed between the fluid jet (58) and the circular wall and cause a fluid flow reversal which increases the resistance to rotation of the fluidized bed upstream of this zone. To minimize the influence thereof, it is preferable for the injection of the thin fluid layer (58) to be parallel to or directed slightly toward the wall of the central duct (3).

FIG. 5 shows an enlargement of the zone around the two injectors (12.1) and (12.2). The solid particles, upstream of the injector (12.1), slide along the plane wall (2.1) along the flow line (41.1). They exert a pressure on the fluid stream (13.1) at its outlet from the injector (12.1), whereof the outlet surface makes an angle (40) of about 90° with the plane surface of the wall (2.2), and they prevent the normal expansion of the fluid entering the reaction chamber, forcing it to follow the flow line (42.1) whereof the pressure compensates for the pressure of the solid particles and diverts them along the flow line (41.2), which progressively penetrates into the layer of this fluid. The solid particles form a barrier, which acts as a more or less permeable deflector according to their concentration, and they confine the fluid between the flow line (42.2) and the polygonal wall (2.2) and the fluid which retains a high average velocity, because it is confined in a narrow space, loses energy and hence pressure as it transfers it to the solid particles flowing along the flow line (41.3), accelerating them, and hence their concentration decreases and their permeability increases, thereby enabling the flow line, (42.3) to deviate from the wall (2.2) and hence enabling the fluid, which has lost some of its energy, to slow down. The flow line (41.4) of the solid particles ultimately runs along the wall (2.2), along which they slide, slow down, and their concentration increases before reaching the next injector (12.2). And so on and so forth.

The concentration of the solid particle stream upstream of the injectors increases as the distance between the fluid injectors (12.1) and (12.2) increases, and hence as their number decreases, and if the surface of the plane wall (2.2) was curved like the walls (2.1) and (2.3) in FIG. 3, they would exert an additional pressure on the solid particle streams (41.1) and (41.4), which would slow them down and increase their concentration and the resistance to the rotation of the fluidized bed.

The angle of deviation (66) between two injectors is smaller if the number of injectors is higher, thereby decreasing the deviation of the solid particle streams (41.2) and (41.3) and hence the pressure exerted on the fluid streams (13.1) and (13.2) and hence also the quantity of solid particles which can concentrate along the polygonal circular wall after having passed through these fluid streams and hence also the resistance to rotation of the fluidized bed. The angle (40) made by the plane of the injector outlet (12.1) and the polygonal circular wall (2.2) is about 90°, enabling the injection of the fluid (13.1) in a direction virtually parallel to this wall (2.2) and thereby increasing the quantity of tangential angular momentum transferred to the solid particles.

This illustration shows that the solid particles are borne by a fluid pad whereof the pressure offsets the centrifugal force and enables these particles to slide along the polygonal circular wall with a very low resistance to rotation, if the number of fluid injectors is high.

The circular reaction chamber can be connected in series with other similar chambers, the outlet (19) of the solid particles from the upstream chamber being connected to the inlet (16) of the next chamber. These circular reaction chambers may be side by side, in the prolongation of one another, or superimposed. They may be inclined or vertical.

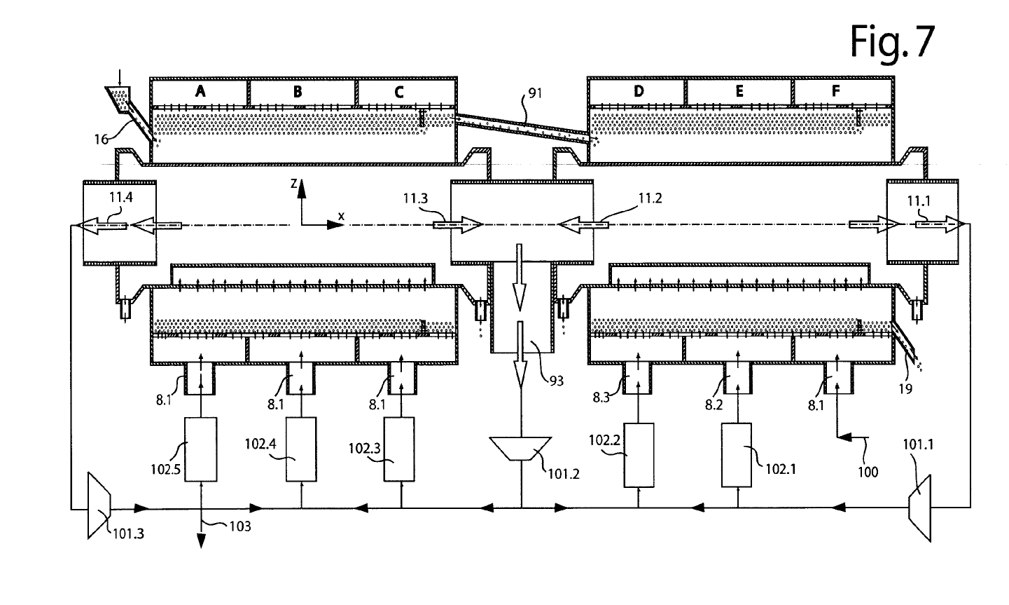

FIG. 6 shows the schematic longitudinal section, in the plane of the (x) and (z) axes, the (z) axis being vertical and coinciding with the axis of rotation (OO′) of the fluidized beds, of the connection of two sections of superimposed circular chambers. The surfaces (18) of the fluidized beds being conical, the fluidized beds of the reaction chambers (6) are subdivided into annular sections by separating rings (80) which support the part of the fluidized bed directly located above them. These are hollow and connected to the fluid distributing chambers (5) via openings (81) for injecting, via the injectors (82), more or less parallel to the plane of the (x) and (y) axes and perpendicular to the axis of rotation (OO′), fluids, symbolized by the arrows (83) in thin layers, which support and rotate the solid particles which bear against the upper part of the separating rings (80).

The separating ring (85) located at the bottom of the reaction chambers is prolonged to the wall of the central duct (3) while the other separating rings (80) have a wide central opening, preferably greater than one-quarter of the average distance between the circular wall and the central duct, to enable the solid particles to pass therein while remaining at a certain distance from the wall of the central duct (3), to avoid being entrained into the central duct via the discharge opening (14).

A stream of solid particles (90) leaves from the bottom of the upper circular reaction chamber via the transfer line (91) which passes through the separating ring (85) and penetrates (92) into the upper part of the lower chamber. The fluid streams (11) are removed from the central ducts (7) by one or more lines (93).

It should be observed that if the fluid pressure beyond the fluidized bed is more or less the same in each circular reaction chamber, the pressure of the inlet of the transfer line (91), located in the fluidized bed, close to the circular wall, is higher than the pressure at its outlet, located outside the fluidized bed, near the wall of the central duct, thereby facilitating the transfer of the solid particles from one reactor to the other, even when the reactors are horizontal and located at the same height.

Finally, the solid particles (95), which have penetrated into the central duct (7) passing through the discharge opening (14) and which fall while rotating in the bottom of the central duct, are removed therefrom by the tube (96), which is actually not in the same plane as the transfer line (90), in order to make them intersect. Since the pressure at this place is lower than the pressure in the reaction chamber, these solid particles must therefore be collected separately, to be optionally recycled by appropriate means.

The separating rings (80) may be replaced by helical turns. The solid particles rotating along the circular wall and along a helical turn will rise if the slope of the turn is upward. In this case, it is possible to transfer the solid particles from the lower chamber to the upper chamber, if the lower part of the transfer line (91) is located along the circular wall, where the pressure is the highest, and the upper part of this line (91) is located against the central duct, where the pressure is the lowest. The particles which are not transferred or removed from the upper part of the circular reaction chamber can fall back into the central space between the inner edge of the turns and the central duct. The helical turns can also be hollow and fed with fluid which is injected along their upper surface into the circular reaction chamber. They can form a continuous or discontinuous helical spiral or may be fragmented into fractions of turns, similar to fixed fins, oriented in the upward direction.

The fluid streams can be recycled according to arrangements adapted to the objectives. For example, FIG. 7 shows an arrangement adapted to the drying of solid particles introduced by the tube (16) on one side of one of the two circular reaction chambers placed in series and exiting via the tube (19) placed at the opposite end of the second chamber, the transfer of these particles from one reactor to the other taking placing via the transfer line (91).

The cool and dry gas (100) is introduced by the tube (8.1) feeding the annular section (F) of the feed chamber located on the side of the outlet (19) of the solid particles. It is heated in contact with the hot solid particles which it cools, while completing their drying before they exit via the tube (19) this gas is then sucked out by the compressor (101.1) through the outlet tube (11.1). It is recycled through treatment units (102.1) and (102.2), for example heat exchangers and/or condensers, by tubes (8.2) and (8.3) to the annular sections (E) and (D). It is then recycled successively by the compressors (101.2) and (101.3) in tubes from (8.3) to (8.6) through treatment units from (102.2) to (102.5), to the annular sections from (D) to (A), in order to progressively remove the moisture from the solid particles. The fluid, which is laden with moisture and which has been cooled by the solid particles, which are introduced by the tube (16) on the side of the tube (8.6) and which it has heated, is removed at (103).

The solid particles may be catalysts which catalyze the chemical conversion of the fluid passing through the fluidized bed. In this case, the fluid is progressively converted. It is in contact during its first passage into the reactor with a spent catalyst which can be regenerated and recycled by appropriate devices, and during its second passage with a fresh or regenerated catalyst, and the treatment units from (102.1) to (102.5) can also serve to remove an undesirable component, for example by absorption or condensation.

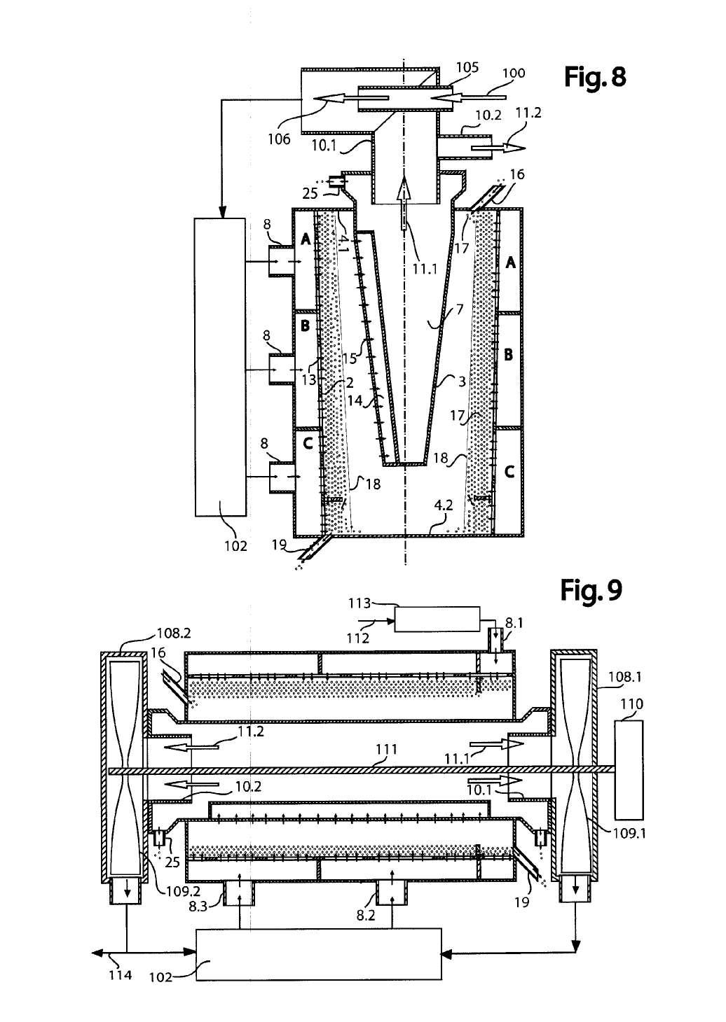

FIG. 8 shows the arrangement of the schematic longitudinal section of a reactor similar to that of FIG. 1, but whereof the axis of rotation of the fluidized bed is vertical or steeply inclined and whereof the central duct (7) terminates at a certain distance above the lower side (4.2). The bottom of the central duct may be closed, as shown in FIG. 8, or open. In this case, the solid particles entering the central duct can be removed therefrom via the bottom during shutdowns, but during operation, swirls may entrain therein the solid particles which accumulate in the bottom of the circular reaction chamber.

This configuration may be advantageous when the quantity of fluid to be removed is not too large. Since the surface (18) of the fluidized bed is conical, very slightly conical in this diagram, implying a very high centrifugal force, the fluid (13) must cross a higher thickness of the fluidized bed in the lower part of the reaction chamber and hence its residence time therein is longer. If this is to be avoided, the circular chamber (2) may also be conical to reduce this difference and/or the quantity of fluid injected into the lower part of the circular reaction chamber can be increased, for example by increasing therein the number and/or cross section of the fluid injectors and/or the pressure in the annular section (C) of the distribution chamber.

FIG. 8 also comprises, for illustration, the arrangement of a system for feeding the fluid by ejector for recycling a fraction of this fluid without using a compressor. This arrangement is useful for when the fluid must be recycled only once or twice and when the use of compressors is difficult, for example due to the corrosiveness of the fluid or very high temperatures, such as, for example, for the dehydrogenation of ethylbenzene or the catalytic cracking of gasoline to light olefins.

The fluid feed (100) optionally preheated, is injected under pressure into an ejector (105), to be injected (106) at very high velocity into the outlet tube (10.1) of the fluid to be recycled (11.1) in order to entrain it into a treatment unit (102), for example a furnace, and to recycle it to the reactor via the tubes (8), before being removed (11.2) via the tube (10.2) toward treatment units.

FIG. 9 shows the diagram of a longitudinal section of a reactor similar to the one in FIG. 1, comprising at each end of the central duct a centrifugal compressor, (108.1) and (108.2), symbolized by the impellers (109.1) and (109.2), which are driven by a common motor (110) using the transmission shaft (111) passing through the central duct. The fresh fluid (112) is fed by the tube (8.1) located on the outlet side (19) of the solid particles, optionally passing through a treatment unit (113) such as for example a moisture condenser. It is then recycled a number of times, successively by the compressors (108.1) and (108.2) through tubes (8.2) and (8.3) and the treatment unit (102), such as for example a heater, before being removed. This very compact arrangement can be advantageously used in easily transportable units, for example, for drying agricultural grains.

The fluid streams can be recycled to the same annular sections, for example to polymerize the catalyst particles in suspension in mixtures of active fluids containing the monomer(s) and possibly having different compositions and/or temperatures from one section to the other, to obtain multimodal and/or wide molecular distribution polymers.

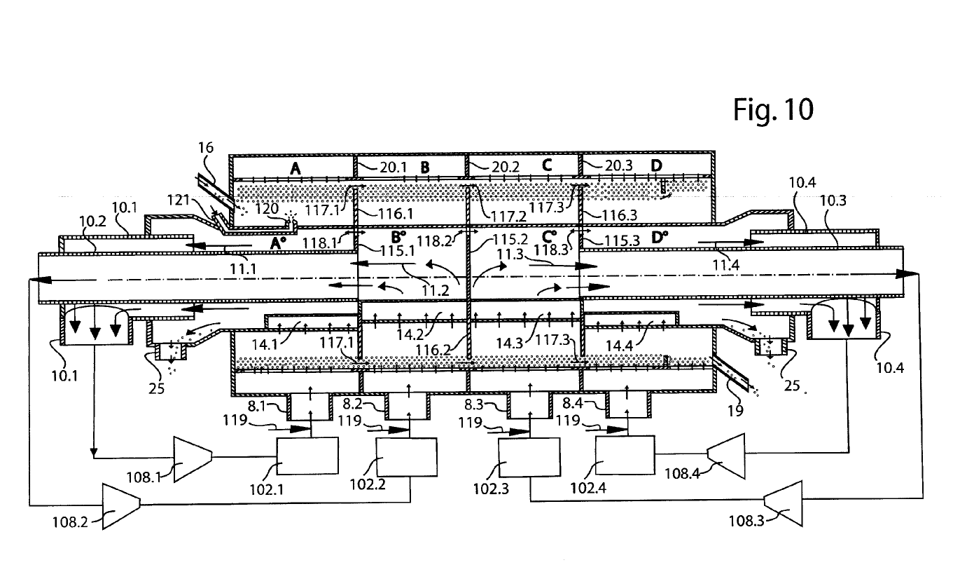

FIG. 10 shows an arrangement which can serve for this type of application. The feed chamber and the central duct are divided into four sections, respectively (A) to (D) and (A°) to (D°), by transverse walls from (20.1) to (20.3) and from (115.1) to (115.3). These walls may be prolonged by annular transverse walls from (116.1) to (116.3) to also separate the circular reaction chamber into four annular sections corresponding to the four sections of the feed chamber and of the central duct, to better separate the fluids from one section to the other, provided that passages from (117.1) to (117.3) are provided in these annular transverse walls, from to (116.1) to (116.3), along the circular wall for transferring the solid particles from one annular section to the other, and passages from (118.1) to (118.3) against the central duct or inside it for the passage of fluid in order to equalize the pressures between the various sections of the central duct.

Four compressors, from (108.1) to (108.4) suck out the fluids from (11.1) to (11.4), from the sections (A°) to (D°), of the central duct through concentric tubes, from (10.1) to (10.4), to recycle it to the feed chambers from (A) to (D), by tubes from (8.1) to (8.4), passing through the treatment units (92.1) to (92.4), for example heat exchangers with optional withdrawal of undesirable components and/or of fluid to be purified before being recycled. The recycled fluids then pass through the rotating fluidized bed and enter the discharge openings of the central duct, from (14.1) to (14.4), to be recycled again to the same sections. The fresh fluids (119) can be fed directly, as required, by the feed tubes from (8.1) to (8.4).

If the fluids are gases, it is possible to spray fine droplets (120) of a liquid on at least part of the surface of the fluidized bed by one or more tubes (121) passing through the central duct.

These arrangements can only operate if the momentum transmitted by the fluid to the solid particles is sufficient to accelerate them as they are transferred from inside the reaction chamber at an average rotational velocity, Vp, which is sufficiently high for the centrifugal force to offset the centripetal pressure exerted by the fluid, and to offset their losses of angular momentum due to the turbulence and the friction along the walls.

It is also necessary for the fluid, after having been slowed down by the solid particles, to retain a sufficient average tangential velocity to avoid a significant reflux. For example, it must make on average at least one half turn before leaving the reaction chamber in the arrangements described above, which contain only one outlet opening (14) per section, and in which the fluid is injected more or less uniformly along the circular wall.

By way of an indicative example, the first condition can be written, for an annular section of the reaction chamber, approximately, ignoring the effect of the pressure variations assumed to be slight on the fluid density:

Ke×m×(Vi−Vt)×Vi×Ei,=Cc×M×p×E×(2×R−E)×Kf×Vp (1)

where
Ke, which may be higher than 1 when the fluid that has been injected is confined between a “wall” of solid particles and the circular wall for converting a fraction of its kinetic energy and/or its pressure to angular momentum, is a variable coefficient of the efficiency of transfer of the tangential angular momentum from the fluid to the particles,
m, Vi and Vt are respectively the mean values of the density, the injection velocity and the tangential velocity of the fluid,
Ei is the sum of the thicknesses (widths) of the outlet openings of the injectors passing through the annular section,
Cc and M are the average concentration and the density of the solid particles,
E and R are the average thickness (width) and the radius of the reaction chamber and,
Kf is a variable friction coefficient representing the % of angular momentum which the solid particles must receive per unit of time to reach and to preserve an average rotational velocity Vp.

Conservation of fluid mass, assuming m constant, which is approximately correct for slight variations in pressure, gives: Ei×Vi≈(1−Cc)×E×Vt/a, where a is the average number of turns or fraction of turns traveled by the fluid before exiting the reaction chamber.

If Vp=β×Vt, where β<1 is a slip coefficient of the solid particles in the fluid, equation (1) becomes: (1−Cc)/a≈Ei/E+X×(2−E/R) (2), where X=p×R×β×Cc×Kf×M/(Ke×m×Vi).

The second condition can be written a>a°, where a°, generally close to ½, is the minimum number of fractions of turns which the fluid must travel on average around the central duct to avoid a reflux permitting the entrainment of an excessive quantity of particles into the duct. Equation (2) then gives: X=p×R×β×Cc×Kf×M/(Ke×m×Vi)<[(1−Cc)/a°−Ei/E]/(2−E/R) (3), and preferably smaller than 1.

This shows that, when the ratio of densities M/m is very high, which is generally the case when the fluid is a gas at a pressure close to atmospheric pressure, the product of the ratios (R/Vi)×(Cc×Kf/Ke) must be very small, requiring a smaller Cc×Kf/Ke ratio and/or a higher fluid injection velocity Vi, the greater the radius R. It is therefore necessary to have a high efficiency of angular momentum transfer from the fluid to the solid particles and low friction between the solid particles and the circular wall to obtain acceptable average solid particle concentrations in industrial-scale reactors using gases at pressures close to atmospheric pressure.

Furthermore, the centrifugal force exerted on the solid particles must also be greater than the centripetal pressure of the fluid, approximately proportional to the square of the average radial velocity, Vr, of the fluid close to the circular wall, to prevent an excessive number of particles from approaching the wall of the central duct (3) upstream of the outlet (14) or of the deflector (40). This can be written, to a first approximation as: Vr<Vc×Vp/(g×R)½ (4); where g is the gravitational acceleration and Vc is the critical upward velocity, which is lower the smaller the size of the solid particles, not to be exceeded in order to obtain a dense fluidized bed, if it is balanced only by the force of gravity.

Conservation of fluid mass, with slight pressure variations which make it possible to ignore the variations in fluid density, gives: 2×p×R×Vr˜E×Vt/a and the inequality (4) becomes approximately: E<2×p×a×β×Vc×(R/g)½<2×a×Vc×(R)½ (5) if R and Vc are expressed in m and m/s.

This inequality indicates that the maximum average thickness of the reaction chamber can only increase proportionally to the square root of R, when the critical velocity, Vc, and hence the size of the solid particles, are very low, and that it is preferable to use small-diameter reaction chambers, if it is undesirable to have a very low E/R ratio.

If it is desirable to have the fluidized bed traversed by a maximum fluid flow when the maximum fluid injection velocity, Vi, is limited, the total cross section, Ei, of the fluid injectors must be increased. If the critical velocity, Vc, is low, the above conditions serve to determine that the optimum is reached when the average thickness (width) of the reaction chamber is approximately: E=2×p×a°×β×Vc×(R/g)½ (6) and that Ei=E×[(1−Cc)/a°−X×(2−E/R)] (7).

Or, as a first approximation, a° generally being close to 0.5 and β close to 1, it is advisable that: E/R<Vc/(R)½ (8) expressed in m and m/s, and Ei/E<2×(1−Cc)−X×(2−E/R) (9) which imposes a low X and hence generally a high injection velocity, Vi, when Vc and hence E/R are low, because the solid particles are small.

However, to avoid approaching the boundary conditions, in practice, it is advisable for estimating the optimum thicknesses (width) of the reaction chamber and of the gas injectors, to use an average concentration, Cc, of the solid particles and/or a theoretical fluid injection velocity, Vi, respectively higher than the solid particle concentrations and lower than the fluid injection velocities which are intended to be used.

### INDICATIVE EXAMPLES

A numerical simulation shows that an average concentration of Cc=30% of very small solid particles, having a critical velocity of Vc=0.4 m/s, can be obtained with a good separation of the fluid and the solid particles, in a reaction chamber 0.4 m in diameter with a central duct 0.14 m in diameter having only one discharge opening, by injecting air at atmospheric pressure at a velocity of 30 m/sec through 8 injectors each having an outlet thickness (width) of 0.004 m, the fluid on average making only one half-turn around the central duct with a fluid residence time in the reactor of about 1/10 second. The estimated average tangential velocity of the solid particles and that of the gas vary respectively from about 4.6 to 4 m/s and from 5.5 to 5 m/s, and the coefficient X and the product of Cc×Kf/Ke vary only from 0.9 to 1 and from 7%/s to 8%/s, when the solid particle concentration is progressively increased from 10 to 30%, confirming that the efficiency of angular momentum transfer from the fluid to the solid particles improves when the solid particle concentration, and hence the “walls” of solid particles channeling the fluid, increases. Losses of solid particles via the central duct appear and increase rapidly when the average solid particle concentration approaches 28% and when the coefficient X is close to 1.

If the number of fluid injectors is reduced to 4, the product Cc×Kf/Ke becomes about 2.5 times higher, imposing an increase in the gas injection velocity Vi to 60 m/sec so that the coefficient X remains below 1 and the losses of solid particles via the central duct become large above a concentration of 25%, which confirms the need to have a large number of gas injectors when the ratio M/m is very high. And if the number of discharge openings in the central duct is increased, the losses of solid particles already become significant with even lower concentrations, confirming the advantage of having only one discharge opening per transverse section of the central duct.

If the ratio of the density of the solid particles to the fluid density is 25 times lower, for example by increasing the pressure to 25 bar, the fluid rotates about 5 times faster and makes on average more than 2 revolutions around the central duct before entering therein, and the centrifugal force is about 25 times higher. This makes it possible to increase the concentration of solid particles and/or decrease the fluid injection velocity and/or increase the diameter of the reaction chamber, while maintaining very good separation of the fluid and the solid particles. The performance can also be improved if the friction coefficient, Kf, is lower and if the efficiency of coefficient of angular momentum transfer, Ke, is higher, which can be obtained by increasing the number of fluid injectors and by improving the profile of the injectors and of the circular chamber.

If a the fluid is a liquid slightly lighter than the solid particles, its number of revolutions, rotational velocity and the centrifugal force further increase, making it possible to preserve acceptable separation of the fluid and the solid particles, even if the critical velocity Vc is much lower due to the slight difference in densities.

These examples show that it is only when the ratio of the density of the solid particles to that of the fluid is several hundreds that the injection velocity of the fluid(s) must be much higher than the desired rotational velocity of the solid particles and/or the reaction chamber must have a small diameter.

The device of the present invention can be applied to industrial processes of catalytic polymerization, drying, impregnation, coating, roasting or other treatments of solid particles in suspension in a fluidized bed, or of cracking, dehydrogenation or other catalytic conversions of fluids or fluid mixtures passing through a fluidized bed.

Example of a Method Using this Device

### Conversion of Cracking Gasolines to Light Olefins

The cylindrical reaction chamber shown in FIG. 8 may, for information, have a diameter of 1 m, a length of 4.5 m and an average thickness (width) of 0.23 m, giving it a volume of about 2.5 m2. The fluid (100), consisting of cracking gasolines preheated to high temperature, having a density of about 5 kg/m³, at the injection temperature and pressure, is injected at high velocity (for example 200 to 300 m/s, giving a potential pressure of 100 to 200 000 Pa) into the ejector (105) to be superheated to the desired temperature (over 600° C.), at the same time as the recycled fluid it entrains into the furnace (102) and then into the reaction chamber, where they are injected, for example, at a velocity of 60 m/s through 17 injection slits 0.005 m thick, giving a flow rate of about 23 m³/s, or 400 tonnes per hour. (This high flow rate requires a central duct passing through the reaction chamber to remove the fluid on both sides, and the reactor may be horizontal or vertical). If the quantity of fluid recycled is about 50%, the cracking gasoline feed flow rate is about 200 tonnes per hour and its average residence time in the reaction chamber is about twice one tenth of a second.

If Cc×Kf×M/m×Ke≈30, which gives X≈0.7, the catalyst powder, which is fed via the tube (16) is entrained by the fluid at an average rotational velocity, Vp, of about 13 m/s, producing a centrifugal force 35 times the force of gravity, generating a pressure on the cylindrical wall of about 30 000 Pa and enabling the fluid to pass through the fluidized bed at a velocity of more than 2 m/s. The catalyst powder is removed via the tube (19) and can be recycled easily after regeneration, with a cycle time which may be between a few minutes and several hours.

### Drying of Agricultural Grains

Grains of agricultural origin can be dried according to the diagram in FIG. 9. The reaction chamber or drying chamber may have the same dimensions as those of the above example. In this case, the fresh air (112) is introduced via the tube (8.1), optionally through a moisture condenser (113), to pass through the end of the reaction chamber on the side of the grain outlet (19) in order to heat them while cooling them and completing their drying. This air (11.1) is then sucked out by the centrifugal compressor or fan (108.1) through the line (10.1) and recycled to the reactor via the line (8.2) after having been heated additionally in the heater (102). After having been recycled several times, this air (11.2) is sucked out by the centrifugal compressor or fan (108.2) through the line (10.2) and recycled to the reactor via the line (8.3) after having been heated by the heater (102). After having again been recycled several times, this moisture laden air cooled by the grains, which are fed via the line (16) and which it has heated, is removed at (114).

The air being sucked out by the compressors or fans, the pressure in the reactor is lower than the atmospheric pressure, which is favorable to drying, and mechanical means can easily transfer the dried grains for storage at atmospheric pressure. The air can be injected into the drying chamber at the same rate of 23 m³/s in the above example, or about 100 tonnes per hour. If it is recycled 5 to 10 times, this gives a fresh air quantity of 10 to 20 tonnes per hour and a contact time with the grains of about 5 to 10 times 0.1 second.

The quantity of grains in the drying chamber may be about 500 kg, giving an average residence time of 90 seconds for drying 20 tonnes per hour, which may be sufficient given the high velocity and low pressure of the air and the possibility of working at higher temperatures thanks to the short residence time and the cooling of the grains before they leave the reactor.

This assembly can be built to be compact and easily transportable, which demonstrates the advantage of having a dense fluidized bed traversed by a very large quantity of fluid at high velocity thanks to the centrifugal force.

### Gas Phase Copolymerization of Ethylene and Octene

The gas phase copolymerization of ethylene and octene is only possible if the pressure in the reactor is low, at a maximum no more than a few times the atmospheric pressure, because the octene partial pressure is limited to about 0.2 bar at 70° C. At these pressures, the quantity of heat produced by these highly exothermic reactions can only be removed by using relatively inactive catalysts or by diluting the mixture of active gases with an inactive gas to slow down the reaction rate, thereby increasing the cost of the installation, or by passing such a large quantity of gas through the fluidized bed as to require a rotating fluidized bed, for example according to the diagram shown in FIG. 10.

The octene can be sprayed in fine droplets (120) into the reaction chamber by the tube (121) passing through the central duct and/or can be fed in gaseous form at the same time as the fresh ethylene (119) and the fluid recycled via one or more of the tubes (8.1) to (8.4). For information, the cylindrical reaction chamber, may, for example, have a diameter of 1.6 m, length 10 m and thickness 0.32 comprising 29 injection slits of thickness 0.005 m, for injecting about 50 m³/s of active fluids, if the fluid injection velocity is 35 m/s. If the pressure is about 3 times the atmospheric pressure, permitting an octene concentration of about 20% by weight, the recycled active fluid flow is about 700 tonnes per hour, suitable for removing the heat of polymerization of about 10 to 20 tonnes per hour of polymer. The quantity of polymer in the reaction chamber of which the volume is about 12 m³ is about 3 tonnes, giving a polymer particle residence time in the reaction chamber of 10 to 15 minutes, making it possible to use highly active catalysts. The rotational velocity of the polymer particles may be about 11 m/s, giving a centrifugal force about 16 times the force of gravity, so that the fluidized bed can be crossed with a radial velocity of more than 1.5 m/s in about 0.2 second.

This reactor can be installed in series, for example after another reactor which may operate at much higher pressures without comonomer or with lighter comonomers, to obtain multimodal polymers. It is also suitable for progressively varying the composition and/or temperature of the fluid passing through the rotating fluidized bed.

### Impregnation or Coating of Solid Particles

The diagram in FIG. 10 can also be used for impregnating or coating solid particles. The fluid serving for impregnation or coating can be sprayed in the form of fine droplets (120) into the part of the reaction chamber located on the feed side of the solid particles by the tube (16). These particles are then dried in the successive annular sections of the circular reaction chamber and the components serving for impregnating or coating the solid particles may even be baked, if the temperature of the recycled fluid is sufficiently high and the solid particles can be recycled by an appropriate device, if it is necessary to apply several coating layers.

1. A rotating fluidized bed device comprising a circular reaction chamber, a device for feeding one or more fluids, placed around the circular wall of said circular reaction chamber, a device for removing said fluid(s), a device for feeding solid particles on one side of said circular reaction chamber and a device for removing said solid particles on the opposite side of said circular reaction chamber, characterized in that:
said device for removing said fluids comprises a central duct passing longitudinally through or penetrating into said circular reaction chamber, the wall of said central duct comprising at least one discharge opening for centrally removing, via said central duct, said fluids from said circular reaction chamber;
said device for feeding said fluids comprises fluid injectors distributed around said circular wall for injecting said fluids in a succession of layers along said circular wall and rotating around said central duct (7) while entraining said solid particles in a rotational movement whereof the centrifugal force thrusts them toward said circular wall through said succession of layers;
said centrifugal force is, on average, at least equal to three times the force of gravity, said solid particles thereby forming a rotating fluidized bed which rotates around and at a certain distance from said central duct while sliding along said circular wall and while being supported by said layers of said fluids which pass through said fluidized bed before being removed centrally via said discharge opening of said central duct and whereof the centripetal force is offset by said centrifugal force exerted on said solid particles.

2. The device of claim 1, characterized in that said discharge openings are arranged longitudinally and in that their average width is less than half of the average distance between said wall of said central duct and said circular wall.

3. The device of claim 1, characterized in that the sum of the cross sections of said discharge opening is smaller than twice the sum of the outlet cross sections of said fluid injectors.

4. The device of claim 1, characterized in that the planes of said discharge openings make angles of between 60° and 120° with the wall of said central duct.

5. The device of claim 1, characterized in that no transverse cross section of said central duct passes through more than one said discharge opening.

6. The device of claim 1, characterized in that the injection directions of the layers of said fluids by said fluid injectors make an angle of less than 30° with said circular wall on the side located downstream of said fluid injectors.

7. The device of claim 1, characterized in that the outlet planes of said fluid injectors make angles of between 60° and 120° with said circular wall on the side located downstream of said fluid injectors.

8. The device of claim 1, characterized in that each annular section of said circular wall contains at least one said fluid injector at 90° intervals.

9. The device of claim 1, characterized in that the distance between two said consecutive fluid injectors is preferably less than the average radius of said circular wall.

10. The device of claim 1, characterized in that the outlets of said fluid injectors are thin, preferably having a width less than one-twentieth of the average radius of said circular reaction chamber.

11. The device of claim 1, characterized in that the surface of said circular wall located between two said consecutive injectors is plane, the circular wall being polygonal.

12. The device of claim 1, characterized in that said device for feeding said fluids comprises a fluid feed chamber surrounding said circular wall, the pressure difference between said fluid feed chamber and said central duct being maintained by said fluids feed and removal devices at more than the average centrifugal pressure exerted by said fluidized bed on said circular wall.

13. The device of claim 12, characterized in that said feed chamber is divided into longitudinal segments by longitudinal walls for feeding said injectors corresponding to said longitudinal segments at different pressures.

14. The device of claim 12, characterized in that said feed chamber is divided into successive annular sections by transverse annular walls for separately feeding said injectors corresponding to each of said successive annular sections and thereby making the corresponding annular sections of said rotating fluidized bed be traversed by fluids having different compositions or temperatures or injection velocities.

15. The device of claim 1, characterized in that said circular reaction chamber is longitudinally traversed by at least one deflector, wing-shaped, close to said central duct upstream of at least one said discharge openings and extending beyond said discharge openings.

16. The device of claim 15, characterized in that said deflector is hollow and is fed with said by said fluid feed device and is equipped with at least one fluid injector along its trailing edge for injecting said fluid, in a thin layer, along the wall of said central duct downstream of said discharge opening.

17. The device of claim 15, characterized in that the distance between said edge located downstream of said hollow deflector and the wall of said central duct located downstream of said discharge opening is less than half the discharge between said edge and said circular wall.

18. The device of claim 1, characterized in that the wall of said central duct is flared at least one of its two ends and in that it comprises a tube for removal of said fluid, concentric with and at a certain distance from said flared wall, and a discharge tube against said flared wall separately removing said solid particles which have been entrained into said central duct and which are thrust by centrifugal force along said flared wall.

19. The device of claim 1, characterized in that said circular reaction chamber is connected to another similar chamber, by a transfer line for transferring said solid particles from said circular reaction chamber to said similar chamber and whereof the inlet is located close to said circular wall of said circular reaction chamber, on the side opposite said device for feeding said solid particles, and whereof the outlet is located close to said central duct of said similar chamber on the side opposite said device for removing said solid particles from said similar chamber.

20. The device of claim 1, characterized in that said circular reaction chamber contains, close to the side of said device for removing said solid particles, a control ring whereof the outer edge extends along and is fixed to said circular wall and whereof the inner edge is at an average distance from said central duct that is higher than one-quarter of the average distance between said central duct and said circular wall said solid particles in suspension in said rotating fluidized bed having to pass into the space located between said inner edge and said central duct to pass from one side of said control ring to the other side.

21. The device of claim 20, characterized in that said control ring comprises at least one passage located against said circular wall for transferring said solid particles located on one side of said control ring toward the other side without having to pass through the space located between said inner edge and said central duct.

22. The device of claim 1, characterized in that said fluids are gases and in that it comprises a liquid injection device, passing via said central duct, for spraying said liquid in fine droplets onto at least part of the surface of said fluidized bed.

23. The device of claim 1, characterized in that said circular reaction chamber contains a set of turns or fractions of helical turns whereof the outer edge extends along and is fixed to said circular wall and whereof the inner edge is at an average distance from said central duct that is greater than one-quarter of the average distance between said central duct and said circular wall.

24. The device of claim 1, characterized in that said device for feeding one or more fluids comprises at last one ejector penetrating into a line for removing said fluids and whereby said feed fluids are injected at very high velocity and mixed with the fluids removed in said removal line for recycling to said circular reaction chamber.

25. The device of claim 1, characterized in that the axis of rotation of said fluidized bed makes an angle of less than 45° with the vertical and in that said central duct passes through the upper side of said circular reaction chamber and terminates at a certain distance on the opposite side, the transverse cross section of said central duct decreasing progressively from the top downward.

26. The device of claim 25, characterized in that the average radius of said circular reaction chamber decreases progressively from the top downward.

27. The device of claim 1, characterized in that the axis of rotation of said fluidized bed makes an angle of less than 45° with the vertical and in that said circular reaction chamber comprises separating rings dividing said rotating fluidized bed into several annular sections, the outer side of said separating rings extending along and being fixed to said circular wall, and their inner edge being at an average distance from said central duct that is greater than one-quarter of the average distance between said central duct and said circular wall, said solid particles in suspension in said rotating fluidized bed having to pass into the space located between said inner edge and said central duct to pass from one side of one of said separating rings to the other side.

28. The device of claim 27, characterized in that said separating rings are hollow and are fed with fluid by said feed device, said fluid being injected in a succession of layers along the upper surface of said rings in the direction of rotation of said rotating fluidized bed.

29. The device of claim 27, characterized in that said separating rings comprise at least one passage, located against said circular wall for allowing the solid particles located above said separating rings to flow downward without having to pass through the space located between said inner edges and said central duct.

30. The device of claim 27, characterized in that the said separating rings are turns or fractions of helical turns, whereof the slope is oriented upward.

31. The device of claim 1, characterized in that the axis of rotation of said fluidized bed makes an angle greater than 45° with the vertical and in that said discharge openings are located on the side of the lower longitudinal part of said circular reaction chamber.

32. The device of claim 15, characterized in that the axis of rotation of said fluidized bed makes an angle greater than 45° with the vertical and in that the leading edge of said deflector is located on the side of the lower longitudinal part of said circular reaction chamber.

33. The device of claim 1, characterized in that it comprises a device for recycling said fluids removed by said device for removing said fluids toward said device for feeding said fluids, said recycling device comprising a device for treating said recycled fluids in order to adjust the temperature or composition thereof.

34. The device of claim 1, characterized in that said central duct is transversely divided by transverse walls in sections connected to discharge tubes placed inside said central duct for separately removing the fluids issuing from said sections of said central duct and for treating and recycling them separately into a corresponding section or another section of said circular reaction chamber.

35. The device of claim 34, characterized in that said circular reaction chamber is divided into annular sections corresponding to said sections of said central duct by annular walls fixed between said circular wall and said central duct these said annular walls comprising at least one passage against said circular wall for the passage of the solid particles from one said annular section toward said adjacent annular section and said annular walls or said transverse walls of said central duct comprising at least one passage located against or in said central duct for the passage of said fluids from one said section to said adjacent section.

36. The device of claim 1, characterized in that it comprises a device for recycling said solid particles removed by said device for removing said solid particles, in order to recycle them to said circular reaction chamber by said device for feeding said solid particles.

37. The device of claim 36, characterized in that said solid particles are catalyst particles and in that said device for recycling said catalytic particles comprises a device for regenerating said catalytic particles.

38. A method of catalytic polymerization, drying or other treatments of solid particles in suspension in a rotating fluidized bed of catalytic conversion of fluids passing through said rotating fluidized bed, characterized in that it comprises the steps which consist in injecting a fluid or fluids in successive layers into a circular reaction chamber, and of removing them centrally via a central duct passing through or penetrating into said circular chamber, as claimed in claim 1, at a flow rate and an injection pressure entraining said solid particles at an average rotational velocity generating a centrifugal force of at least three times greater than the force of gravity.

39. A method of catalytic polymerization, drying or other treatments of solid particles in suspension in a rotating fluidized bed or of catalytic conversion of fluids passing through said rotating fluidized bed, as claimed in claim 38, wherein said method comprises recycling said fluid or fluids, wherein said recycling comprises a device for treating said recycled fluids in order to adjust the temperature or composition thereof.

40. A method of catalytic polymerization, drying or other treatments of solid particles in suspension in a rotating fluidized bed or of catalytic conversion of fluids passing through said rotating fluidized bed, as claimed in claim 38, characterized in that it comprises the step which consists in recycling said solid particles to said circular reaction chamber.

41. A method of catalytic polymerization, impregnating, coating or other treatments of solid particles in suspension in a rotating fluidized bed, as claimed in claim 38, characterized in that it comprises the steps which consist in spraying a liquid in fine droplets onto said solid particles and in making said liquid impregnating or surrounding said particles react chemically with said gaseous fluid or fluids passing through said rotating fluidized bed.

42. A method of polymerization comprising the device described in claim 1.

43. The method of claim 42, wherein at least one of said fluids contains alpha-olefins.

44. A method of catalytic conversion of a fluid or fluid mixture passing through a rotating fluidized bed whereof the solid particles are catalysts comprising the device of claim 1.

45. The method of claim 44, wherein said fluid or mixture of fluids contains olefins and in that said catalytic conversion involves changing the distribution of the molecular weights of said olefins.

46. The method of claim 44, wherein said fluid or mixture of fluid contains ethylbenzene and in that said catalytic conversion involves its dehydrogenation in order to convert it to styrene.

47. The method of claim 46, wherein said solid particles contain components that can react with the hydrogen coming from said dehydrogenation, so as to reduce the concentration thereof in the fluid or mixture of fluids, it being possible for these said components to be regenerated outside said circular reaction chamber.

48. A method for drying or extracting volatile components from said solid particles comprising the device of claim 1.

49. A method for impregnating or coating said solid particles comprising the device of claim 1.

50. The method of claim 48, wherein said solid particles are grains, powder or other fragments of agricultural origin.

51. The method of claim 49, wherein said solid particles are grains, powder or other fragments of agricultural origin.
